(19) United States
(12) Patent Application Publication
(10) Pub. No.: US 2007/0240985 A1
(43) Pub. Date: Oct. 18, 2007

(54) DEVICE USED TO DETECT NUCLEIC ACID

(76) Inventors: Jun Okada, Tokyo (JP); Sadato Hongo, Yokohama-shi (JP); Nobuhiro Gemma, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/734,074

(22) Filed: Apr. 11, 2007

(30) Foreign Application Priority Data
Apr. 13, 2006 (JP) 2006-111178

(51) Int. Cl. G01N 33/487

(52) U.S. Cl. 204/403.01

(57) ABSTRACT

A device includes a flow channel through which a solution containing at least one of a target nucleic acid and a nucleic acid recognition body flows. The flow channel includes a flat bottom surface parallel to the flowing direction of the solution and an inclined surface gradually projecting from the bottom surface in the flowing direction. The device also includes a probe electrode provided on the inclined surface. The probe electrode is formed of an exposed portion of a metal film through an opening of a passivation film stacked on the metal film. An angle θ that the probe electrode forms with respect to the bottom surface, a maximal width R of the opening in the flowing direction, and a thickness d of the passivation film satisfy tan θ>d/R.

### CROSS-REFERENCE TO RELATED APPLICATIONS

This application is based upon and claims the benefit of priority from prior Japanese Patent Application No. 2006-111178, filed Apr. 13, 2006, the entire contents of which are incorporated herein by reference.

### BACKGROUND OF THE INVENTION

1. Field of the Invention

The present invention relates to a device used to detect a nucleic acid utilizing an electrochemical response.

2. Description of the Related Art

As genetic engineering develops in recent years, disease diagnosis or prevention using a gene has become possible in the medical field. This is called gene diagnosis. Detecting a human gene defect or change that causes a disease allows the disease to be diagnosed or predicted precritically or at its very early stage. Along with human genome decoding, researches on the relationship between the genotype and disease advance. Treatments that match the genotype of the individual patients (tailor-made medical care) are being put into practice. Accordingly, it is very important to detect a gene and determine a genotype simply.

Examples of nucleic acid detection methods include a method using a radioisotope and a method using a fluorescent dye label. The former method can perform detection only at limited locations and requires cumbersome operation. The latter method requires an expensive apparatus to detect a fluorescent dye.

Besides these techniques, another technique has been established. According to this technique, a sample nucleic acid is hybridized with a nucleic acid probe immobilized to the surface of an electrode. Then, a nucleic acid recognition body is added a nucleic acid is electrochemically detected. The technique of electrochemically detecting the nucleic acid is suitable to “Lab-on-a-chip” that causes reactions on a single chip. Hence, this technique has been under development in a variety of applications. Recently, to improve the sensitivity in the technique of electrochemical nucleic acid detection, a technique using an electrode with a small area has been proposed.

Electrical detection requires an electrode. Various techniques are available to form an electrode simply on a chip. One example of such techniques uses a passivation film. According to this technique, a passivation film with a hole is formed on a metal film which is formed on a substrate to serve as an electrode. The opening of the passivation film defines an electrode area. This technique allows formation of an electrode with a uniform thickness or a uniform surface state on a chip comparatively easily.

Considering formation of an electrode with a small area in order to perform the sensitivity improving technique described above, when the thickness of the passivation film and the diameter of the electrode are almost equal, the influence that the wall of a passivation film existing around the electrode causes on the reaction of the electrode is large. As the reaction of the nucleic acid occurs in a liquid layer, the wall of the passivation film hinders a solution containing at least one of a target nucleic acid and a nucleic acid recognition body from readily reaching the electrode.

### BRIEF SUMMARY OF THE INVENTION

The present invention is directed to a device used to detect a nucleic acid utilizing an electrochemical response. This nucleic acid detection device comprises a flow channel through which a solution containing at least one of a target nucleic acid and a nucleic acid recognition body flows, and a probe electrode provided in the flow channel. The probe electrode is formed of an exposed portion of a metal film through an opening of a passivation film stacked on the metal film.

In one nucleic acid detection device according to the present invention, the flow channel includes a flat bottom surface parallel to the flowing direction of the solution, and an inclined surface gradually projecting from the bottom surface in the flowing direction. The probe electrode is arranged on the inclined surface. An angle θ that the probe electrode forms with respect to the bottom surface, a maximal width R of the opening in the flowing direction, and a thickness d of the passivation film satisfy tan θ>d/R.

In another nucleic acid detection device according to the present invention, the passivation film satisfies d2>d1 where d1 is a thickness of the passivation film upstream in the flowing direction of the solution, and d2 is a thickness of the passivation film downstream in the flowing direction of the solution.

In still another nucleic acid detection device according to the present invention, the flow channel includes a flat bottom surface parallel to the flowing direction of the solution, and a projection including a projecting surface projecting from the flat bottom surface. The probe electrode is arranged on the projecting surface. A distance L from an end of the projection upstream in the flowing direction of the solution to the probe electrode satisfies L<0.065×Re×D, Re=ρuD/μ, and D=4S/Lp where ρ is the density of the solution, u is the flow velocity of the solution, μ is the viscosity of the solution, S is the sectional area of the flow channel, and Lp is the wall peripheral perimeter of the flow channel.

In still another nucleic acid detection device according to the present invention, the passivation film includes a first portion which covers an electrode area surrounding the probe electrode, and a second portion which covers an area different from the electrode area. The first portion is thinner than the second portion.

Still another nucleic acid detection device according to the present invention further includes a planarization layer provided under the metal film and the passivation film and including a flat upper surface.

### BRIEF DESCRIPTION OF THE SEVERAL VIEWS OF THE DRAWING

FIG. 1 shows a nucleic acid detection device;

FIG. 2 shows a probe electrode shown in FIG. 1 in enlargement;

FIG. 3 shows a section of the probe electrode and its peripheral portion taken along the line III-III of FIG. 2;

FIG. 4 schematically shows the flow of a solution in a flow channel;

FIG. 5 shows a section of a probe electrode and its peripheral portion in a nucleic acid detection device according to the first embodiment;

FIG. 6 schematically shows the section shown in FIG. 5;

FIG. 7 shows a section of a probe electrode and its peripheral portion in a nucleic acid detection device according to the second embodiment;

FIG. 8 schematically shows the section shown in FIG. 7;

FIG. 9 shows a section of a probe electrode and its peripheral portion in a nucleic acid detection device according to the third embodiment;

FIG. 10 shows a perspective of the structure shown in FIG. 9;

FIG. 11 schematically shows the section shown in FIG. 9;

FIG. 12 shows another nucleic acid detection device;

FIG. 13 shows a section of an electrode area and of a circuit area in the nucleic acid detection device shown in FIG. 12;

FIG. 14 shows a section of an electrode area and of a circuit area in a nucleic acid detection device according to the fourth embodiment;

FIG. 15 shows a section of an electrode area and of a circuit area in a nucleic acid detection device according to the fifth embodiment; and

FIG. 16 shows results obtained by performing electrochemical measurement in an example.

### DETAILED DESCRIPTION OF THE INVENTION

The embodiments will be described with reference to the accompanying drawing.

### First Embodiment

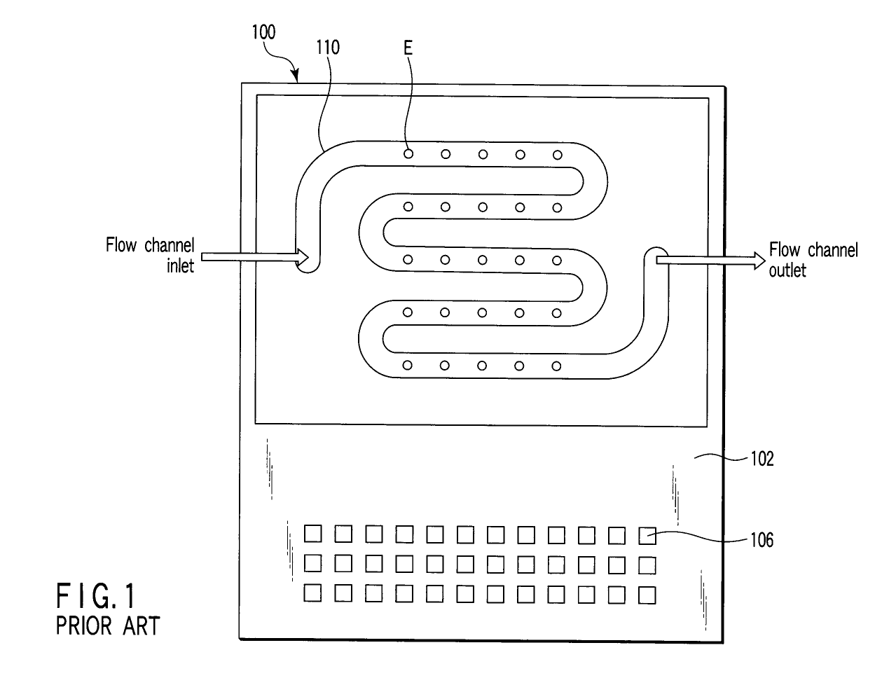

As shown in FIG. 1, a nucleic acid detection device 100 includes a support substrate 102, signal input/output pads 106 provided to the support substrate 102, a flow channel 110 formed on the support substrate 102, and probe electrodes E provided in the flow channel 110.

The flow channel 110 is for carrying a solution containing at least one of target nucleic acids and nucleic acid detection bodies. The probe electrodes are arranged at intervals to form a row along the flow channel 110. Although not shown, the nucleic acid detection device 100 has, in addition to the probe electrodes, a counter electrode to measure an electrochemical reaction, and preferably a counter electrode and a reference electrode, in the flow channel 110. The counter electrode and reference electrode are respectively electrically connected to the signal input/output pads 106 through wiring lines formed in or on the surface of the nucleic acid detection device 100.

The nucleic acid detection device 100 is used as it is mounted on a known nucleic acid detection apparatus. The nucleic acid detection apparatus supplies the solution to the flow channel 110 and measures the current flowing through the electrodes through the signal input/output pads 106.

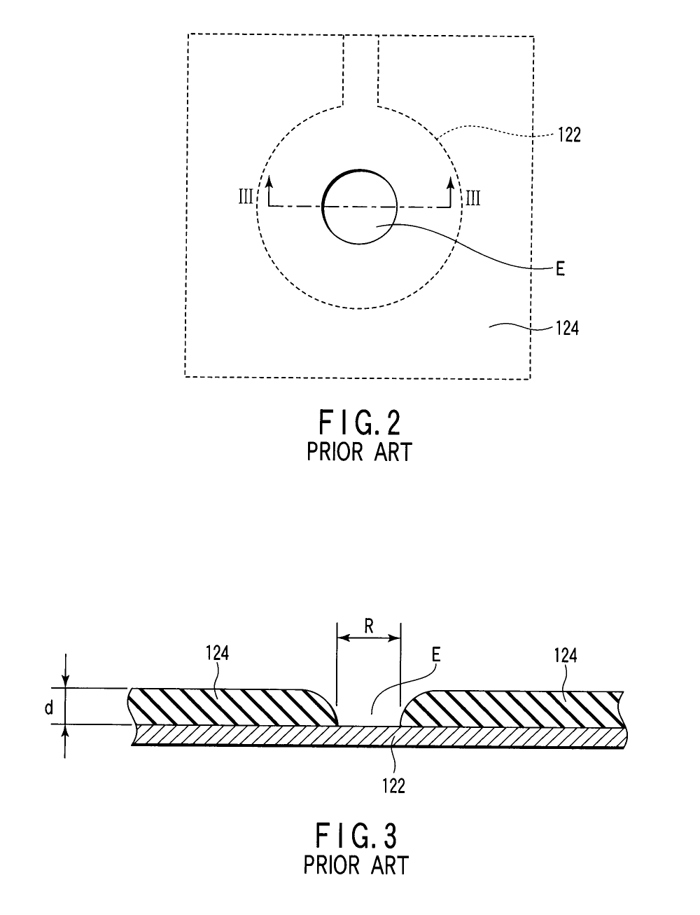

As shown in FIGS. 2 and 3, a passivation film 124 is stacked on a metal film 122 patterned to form a probe electrode. The passivation film 124 has an opening to partially expose the metal film 122. That portion of the metal film 122 which is exposed through the opening of the passivation film 124 constitutes the probe electrode E. Namely, the nucleic acid detection device 100 has the metal film 122 including the probe electrode E, and the passivation film 124 provided with the opening that defines the probe electrode E.

Referring to FIG. 4, the length of each arrow reflects the velocity of the flow of the solution at that position. The flow of the solution in the flow channel is stable, and the solution flows almost parallel to the flow channel. In the stable flow, as is apparent from FIG. 4, the solution flows slow near the wall surface of the flow channel 110, i.e., near the passivation film 124, and faster at a position farther away from the wall surface of the flow channel 110, i.e., from the passivation film 124.

The smaller the area of the probe electrode E, the higher for sensitivity. In other words, the smaller the maximal width of the probe electrode E, i.e., a diameter R for a circular probe electrode, the better. When, however, the diameter R of the probe electrode E is almost equal to a thickness d of the passivation film 124, in the stable flow described above, the wall of the passivation film 124 existing around the probe electrode E hinders the solution from smoothly entering the opening, so that the solution dose not reach the surface of the probe electrode E easily. Accordingly, difficulty in detection increases. Such a phenomenon appears when the diameter R of the nucleic acid detection probe immobilization electrode E is smaller than about 100 times the thickness d of the passivation film 124.

This embodiment is directed to a structure that allows the solution to easily reach even a probe electrode E with a small area.

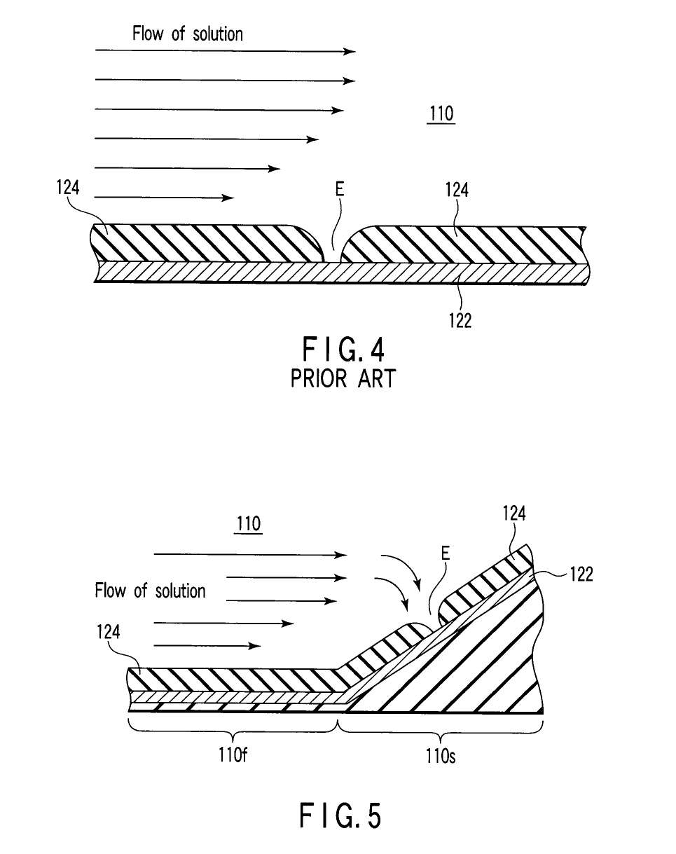

As shown in FIG. 5, the flow channel 110 has a flat portion 110f having a flat bottom surface parallel to the designed flowing direction of the solution, and an inclined portion 110s having an inclined surface gradually projecting from the flat bottom surface in the flowing direction of the solution. The probe electrode E is arranged on the inclined surface of the inclined portion 110s.

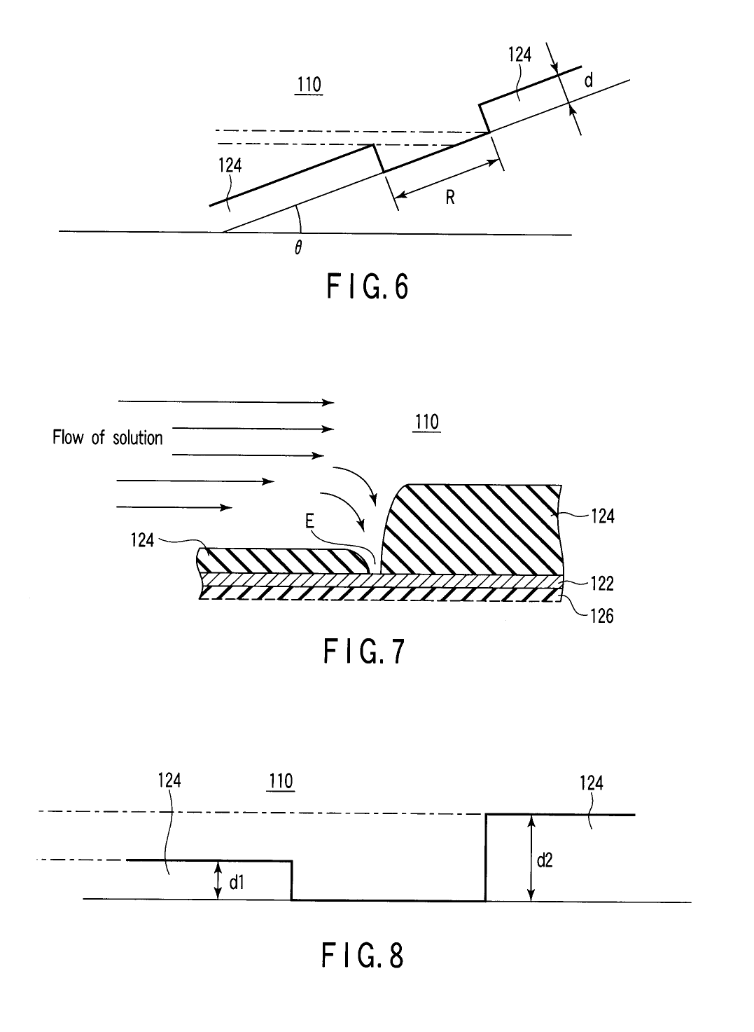

As shown in FIG. 6, an angle θ that the probe electrode E forms with respect to the flat bottom surface of the flat portion 110f, the maximal width R of the opening of the passivation film 124 in the flowing direction of the solution, and the thickness d of the passivation film 124 satisfy tan θ>d/R. As far as this inequality is satisfied, a straight line that is parallel to the designed flow of the solution and crosses the metal film 122 without crossing the passivation film 124 exists, as is apparent from FIG. 6. Hence, the flowing solution can reach the probe electrode E directly. Consequently, at least one of a target nucleic acid and a nucleic acid recognition body contained in the solution is supplied to the probe electrode E.

The nucleic acid detection device according to this embodiment is fabricated in the following manner.

A silicon substrate is made with the anisotropic etching to form structures on the substrate surface such that an area around the electrode forms an inclined surface. The material of the substrate is not limited to silicon, but semiconductor, glass, resin, ceramics, metal, or the like may be employed. The inclined surface may alternatively be formed by etching, sputtering, deposition, cutting, molding, printing, or the like. The inclined surface is formed so that the angle θ of the inclined surface with respect to the flat bottom surface satisfies tan θ>d/R with respect to the designed supplying direction of the solution of the flow channel. After that, a metal thin film is formed by sputtering. Alternatively, the metal thin film may be formed by deposition, printing, or the like. After that, wiring portions, pad portions, and the like are patterned by etching where necessary. After that, a passivation film is formed by coating. Alternatively, the passivation film may be formed by sputtering, deposition, printing, or the like. After that, the passivation film is selectively etched to form openings, so the metal thin film is partly exposed through the openings to serve as electrodes. The technique to form the openings is not particularly specified.

Subsequently, by dropping a solution containing nucleic acid probes onto the electrodes, the nucleic acid probes are immobilized on the electrodes to form probe electrodes. Alternatively, the nucleic acid probes may be immobilized by adsorption. By dropping different types of solutions containing different types of nucleic acid probes onto different electrodes, a device for detecting different nucleic acids can be formed.

A component having a preformed flow channel structure is adhered or contact-bonded to the structure formed in this manner to finish a nucleic acid detection device. The electrode forming step and nucleic acid probe immobilization step described above may be applied to a structure that is etched or cut in advance into a flow channel shape.

The nucleic acid detection device according to this embodiment is used in the following manner.

A solution containing target nucleic acids is supplied to the flow channel to cause a hybridization reaction. As the nucleic acid detection device according to this embodiment has an inclined portion in an area around each electrode and the angle θ of the inclined surface with respect to the flat bottom surface satisfies tan θ>d/R, the target nucleic acids contained in the solution are supplied to the probe electrodes efficiently. Thus, even a small amount of nucleic acids sufficiently allows a hybridization reaction. In contrast to this, if tan θ>d/R is not satisfied, the flowing solution cannot directly collide against the probe electrodes. Hence, the target nucleic acids contained in the solution are not supplied to the probe electrodes efficiently.

A washing buffer is supplied to the flow channel to clean the flow channel. As tan θ>d/R is satisfied, the washing buffer is supplied to the probe electrodes efficiently, to clean the probe electrodes reliably.

A solution containing nucleic acid recognition bodies is supplied to the flow channel to cause the hybridized nucleic acids to react with the nucleic acid recognition bodies. As tan θ>d/R is satisfied, the nucleic acid recognition bodies are supplied to the probe electrodes efficiently, to cause the reaction reliably.

The electrochemical response of the nucleic acid recognition bodies is measured to detect the nucleic acids.

### Second Embodiment

The basic structure of a nucleic acid detection device according to the second embodiment is the same as that of the first embodiment. This embodiment is directed to another structure that allows a solution to easily reach even a probe electrode E with a small area as well.

As shown in FIG. 7, a passivation film 124, which defines the probe electrode E, is thin and thick on the upstream side and the downstream side, respectively, in the designed flowing direction of the solution. In other words, as shown in FIG. 8, the passivation film 124 satisfies d2>d1 where d1 is an upstream thickness and d2 is a downstream thickness. As far as this inequality is satisfied, a straight line that is parallel to the designed flow of the solution and crosses the downstream passivation film 124 without crossing the upstream passivation film 124 exists, as is apparent from FIG. 8. Hence, the flowing solution reaches the wall surface of the downstream passivation film 124 directly. This consequently disorders the flow, so at least one of a target nucleic acid and a nucleic acid recognition body contained in the solution is supplied to the probe electrode E.

The nucleic acid detection device according to this embodiment is fabricated in the following manner.

A metal thin film is formed on a substrate by sputtering. Alternatively, the metal thin film may be formed by deposition, printing, or the like. After that, wiring portions, pad portions, and the like are patterned by etching where necessary. After that, a passivation film is formed by coating. Alternatively, the passivation film may be formed by sputtering, deposition, printing, or the like. After that, the passivation film is selectively etched to form openings, so the metal thin film is partly exposed through the openings to serve as electrodes. The technique to form the openings is not particularly specified. At this time, the passivation film is formed to satisfy d2>d1 with respect to the supplying direction of the solution of the flow channel which is designed in advance.

After that, nucleic acid probes are immobilized and a flow channel is formed in the same manner as in the first embodiment.

The nucleic acid detection device according to this embodiment is used in the following manner.

A solution containing target nucleic acids is supplied to the flow channel to cause a hybridization reaction. As the passivation film of the nucleic acid detection device according to this embodiment satisfies d2>d1, the target nucleic acids contained in the solution are supplied to the probe electrodes efficiently. Thus, even a small amount of nucleic acids sufficiently allows a hybridization reaction. In contrast to this, if the passivation film does not satisfy d2>d1, the flowing solution is not sufficiently supplied to the probe electrodes. Hence, the target nucleic acids contained in the solution are not supplied to the probe electrodes efficiently.

After that, washing, reaction with the nucleic acid recognition bodies, and measurement of an electrochemical reaction are performed in the same manner as in the first embodiment to detect nucleic acids.

### Third Embodiment

The basic structure of a nucleic acid detection device according to the third embodiment is the same as that of the first embodiment. This embodiment is directed to still another structure that allows a solution to easily reach even a probe electrode E with a small area as well.

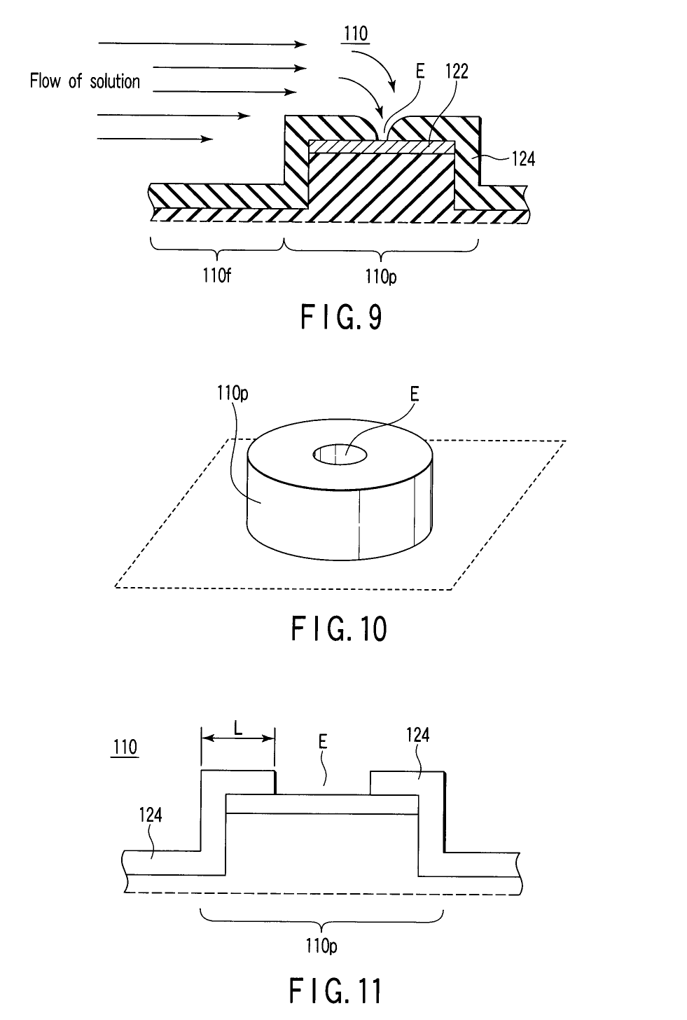

As shown in FIG. 9, a flow channel 110 has a flat portion 110f having a flat bottom surface parallel to the designed flowing direction of the solution, and a projection 110p having a projection 110p which projects from the flat bottom surface and has a projection surface parallel to the flat bottom surface in FIG. 9. As shown in FIGS. 9 and 10, the probe electrode E is arranged on the projection surface of the projection 110p.

As shown in FIG. 11, a distance L from an end of the projection 110p which is upstream in the designed flowing direction of the solution to the probe electrode E satisfies L<0.065×Re×D where Re=ρuD/μ and D=4S/Lp, and ρ is the density of the solution, u is the flow velocity of the solution, μ is the viscosity of the solution, S is the sectional area of the flow channel 110, and Lp is the wall peripheral perimeter of the flow channel 110.

In the nucleic acid detection device according to this embodiment, the flow of the solution is a laminar flow until reaching the projection 110p. When colliding against the projection 110p, the laminar flow changes to a turbulent flow, and thereafter is restored to the laminar flow. The right-hand side of the above inequality is known to express a distance through which the flow of a fluid changes from a turbulent flow to a laminar flow. Accordingly, as far as the distance L satisfies the above inequality, the flow of the solution that has collided against the upstream end of the projection 110p and transformed into a turbulent flow reaches each probe electrode E in the form of the turbulent flow. This supplies at least one of the target nucleic acid and nucleic acid recognition body contained in the solution to the probe electrode E.

The upper limit of the distance L was determined on the basis of the following consideration.

When the solution flows in from a tube inlet at a uniform velocity, a boundary layer develops along the tube wall. When the boundary layer increases its thickness and reaches the center, the velocity distribution in the tube becomes constant (e.g., a parabolic velocity distribution). The region until reaching the fully developed flow is called an entrance region, and its length is called an inlet length X.

For a laminar flow, X/D is a function of Re. According to Boussinesq, X/D≧0.065×Re.

The magnification of a dimensionless number Re which is defined by the following inequality and named Reynolds number serves to discriminate whether an in-tube flow is a laminar flow or a turbulent flow.

Re=ρul/μ

where l is the typical length of the flow, u is the typical flow velocity, ρ is the density of the fluid, and u is the viscosity of the fluid. As the number Re is a dimensionless number, its value is the same regardless of the employed system of units as far as the state of the flow is the same. For a flow in the tube, Re=ρuD/μ is employed by using an inner diameter D of the tube to substitute the typical length l of the flow.

When the section of the pipeline is not circular, turbulent flow transition, pressure loss, and the like can be dealt with in the same manner as a circular cylindrical tube if employing an equivalent diameter De which is defined by the following inequality as the typical length of Re:

De=4S/lp

where S is the sectional area of the flow, and lp is the perimeter of the periphery of the solid wall with which the flow is in contact, i.e., a wetted perimeter.

In the nucleic acid detection device according to this embodiment, if the distance L is smaller than the entrance distance X, i.e., if L<X, the turbulent flow generated at the upstream end of the projection 110p reaches the probe electrode E before being restored to a laminar flow. Substitution of X=0.065×Re×D for L<X yields the relationship described above. In this case, a wall peripheral perimeter Lp of the flow channel substitutes for the wetted perimeter lp in accordance with the structure of the nucleic acid detection device.

The nucleic acid detection device according to this embodiment is fabricated in the following manner.

A substrate is etched or cut to form structures on the substrate surface such that an area around each electrode projects from the substrate surface. The material of the substrate is not limited to silicon, but semiconductor, glass, resin, ceramics, metal, or the like may be employed. After that, a metal thin film is formed on the substrate by sputtering. Alternatively, the metal thin film may be formed by deposition, printing, or the like. After that, wiring portions, pad portions, and the like are patterned by etching where necessary. After that, a passivation film is formed by coating. Alternatively, the passivation film may be formed by sputtering, deposition, printing, or the like. After that, the passivation film is selectively etched to form openings, so the metal thin film is partly exposed through the openings to serve as electrodes. The technique to form the openings is not particularly specified. At this time, the openings are formed to satisfy L<0.065×Re×D with respect to the designed supplying direction of the solution of the flow channel.

After that, nucleic acid probes are immobilized, and a flow channel is formed, in the same manner as in the first embodiment.

The nucleic acid detection device according to this embodiment is used in the following manner.

A solution containing target nucleic acids is supplied to the flow channel to cause a hybridization reaction. The nucleic acid detection device according to this embodiment has a projection in an area around each electrode, and the distance L from the upstream end of the projection to the probe electrode satisfies L<0.065×Re×D. Thus, the flow of the solution collides against the upstream end of the projection to form a turbulent flow, and reaches the probe electrode before being restored to a laminar flow. Thus, the target nucleic acids contained in the solution are supplied to the probe electrodes efficiently. Thus, even a small amount of nucleic acids sufficiently allows a hybridization reaction. If the flow of the solution forms a laminar flow, the velocity decreases in the vicinity of the wall surfaces of the flow channel, i.e., on the probe electrodes, and the target nucleic acids contained in the solution are not supplied to the probe electrodes efficiently.

After that, washing, reaction with the nucleic acid recognition bodies, and measurement of an electrochemical reaction are performed in the same manner as in the first embodiment to detect nucleic acids.

### Fourth Embodiment

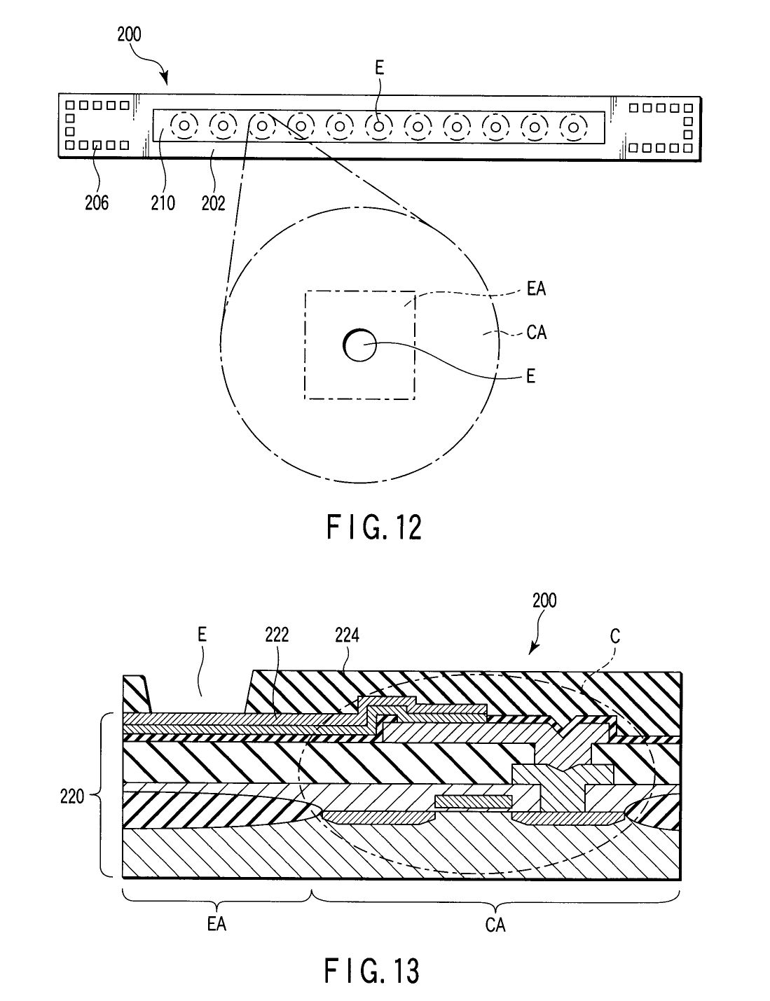

As shown in FIG. 12, a nucleic acid detection device 200 has a support substrate 202, signal input/output pads 206 provided to the support substrate 202, a flow channel 210 formed on the support substrate 202, and probe electrodes E provided in the flow channel 210.

The flow channel 210 is for carrying a solution containing at least one of target nucleic acids and nucleic acid detection bodies. The probe electrodes E are arranged at intervals to form a row along the flow channel 210.

The nucleic acid detection device 200 incorporates a circuit which measures an electrochemical response of the nucleic acid recognition body for nucleic acid detection. The circuit is formed in a circuit area CA located around an electrode area EA where a metal film patterned to form a probe electrode is arranged.

As shown in FIG. 13, a passivation film 224 is stacked on a metal film 222 patterned to form a probe electrode. The passivation film 224 has an opening to partially expose the metal film 222. That portion of the metal film 222 which is exposed through the opening of the passivation film 224 constitutes the probe electrode E. Namely, the nucleic acid detection device 200 has the metal film 222 including the probe electrode E, and the passivation film 224 stacked on the metal film 222 and provided with the opening that defines the probe electrode E.

The metal film 222 electrically is connected to a circuit C built in the circuit area CA. In a structure 220 before forming the passivation film 224, usually, the surface of the electrode area EA is desirably flat, but the surface of the circuit area CA may have many steps.

To improve the measurement sensitivity of the electrochemical response, the probe electrode E is preferably small. To supply at least one of the target nucleic acid and nucleic acid recognition body in the solution to the probe electrode E efficiently, the thinner the passivation film 224, the better. As the surface of the circuit area CA has the many steps, if the passivation film 224 is thin, the corners of the steps tend to be exposed readily, and it is difficult to reliably cover the circuit area CA. Accordingly, the passivation film 224 must be thick to reliably cover the circuit area CA. The thick passivation film 224, however, interrupts at least one of the target nucleic acid and nucleic acid recognition body in the solution from reaching the probe electrode E.

This embodiment is directed to a structure that allows the solution to readily reach even a probe electrode E with a small area while reliably covering the steps.

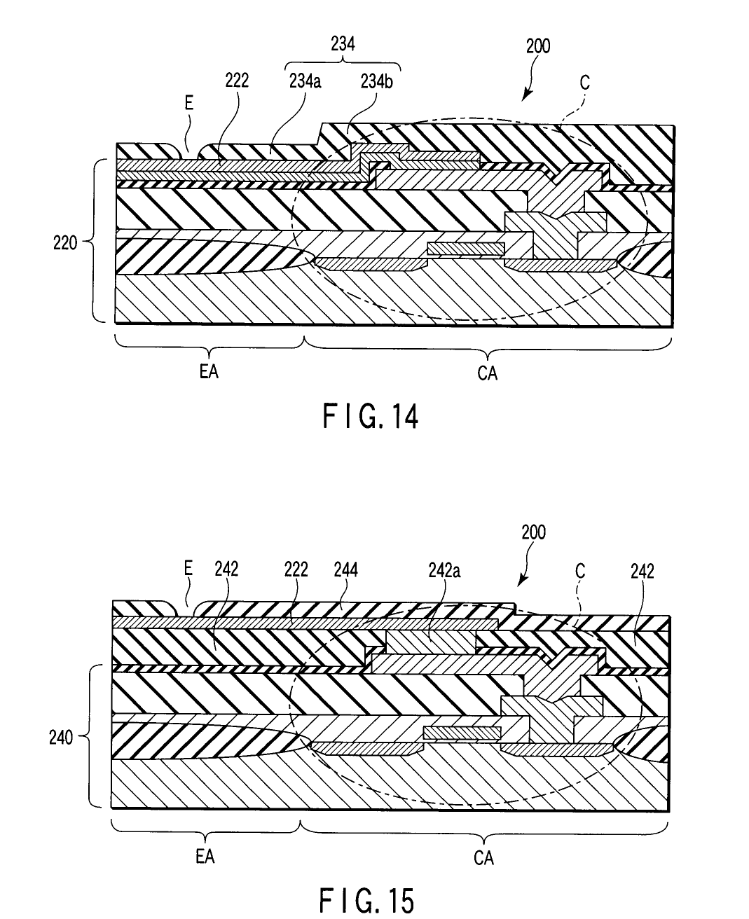

As shown in FIG. 14, the circuit C is built in the circuit area CA, and a passivation film 234 is formed on the surface of the structure 220 having the metal film 222 which is patterned to form the probe electrode. The passivation film 234 includes a portion 234a which covers the electrode area EA and a portion 234b which covers the circuit area CA. The portion 234a which covers the electrode area EA is thinner than the portion 234b which covers the circuit area CA. The thin portion 234a of the passivation film 234 which covers the electrode area EA has an opening that defines the probe electrode E.

The thick portion 234b of the passivation film 234 reliably covers the steps on the surface of the circuit area CA of the structure 220. In addition, as the portion 234a present around the probe electrode E is thin, the solution can reach the probe electrode E readily.

The nucleic acid detection device according to this embodiment is fabricated in the following manner.

A metal thin film is formed on a semiconductor substrate including a circuit, or a substrate having steps and made of glass, resin, ceramics, metal, or the like by sputtering. Alternatively, the metal thin film may be formed by deposition, printing, or the like. After that, wiring portions, pad portions, and the like are patterned by etching where necessary. After that, a passivation film is formed by coating. Alternatively, the passivation film may be formed by sputtering, deposition, printing, or the like. After that, the passivation film is selectively etched to form openings, so the metal thin film is partly exposed through the openings to serve as electrodes. The technique to form the openings is not particularly specified. At this time, the passivation film is formed such that a portion of it which covers each electrode area EA becomes thinner than a portion of it which covers the circuit area CA. The procedure to form the passivation film is not particularly specified. For example, the passivation film can be obtained by forming a comparatively thin first passivation film, forming openings that define electrodes in the comparatively thin first passivation film, forming a comparatively thick second passivation film, and forming openings larger than those in the first passivation film in the second passivation film. According to another procedure, the passivation film can be obtained by forming both first and second passivation films, forming openings larger than electrodes in the second passivation film, and thereafter forming openings that define the electrodes of the first passivation film. According to still another procedure, the passivation film can be obtained by forming a second passivation film first, forming openings larger than the electrodes in the second passivation film, thereafter forming a first passivation film, and forming openings that define the electrodes in the first passivation film. The first and second passivation films can be of the same type or different types. Three or more types of passivation films can be used. This structure can decrease the thickness of the first passivation film that defines the electrodes. At this time, to cover portions having large steps, a passivation film having a large thickness may be formed.

After that, the nucleic acid probes are immobilized to form the probe electrodes in the same manner as in the first embodiment.

The nucleic acid detection device according to this embodiment is used in the following manner.

A solution containing target nucleic acids is supplied to the flow channel to cause a hybridization reaction. As the passivation film which defines the probe electrodes is thin, the target nucleic acids contained in the solution are supplied to the probe electrodes efficiently. Thus, even a small amount of nucleic acids sufficiently allows a hybridization reaction.

After that, washing, reaction with the nucleic acid recognition bodies, and measurement of an electrochemical reaction are performed in the same manner as in the first embodiment to detect nucleic acids.

### Fifth Embodiment

The basic structure of a nucleic acid detection device according to the fifth embodiment is the same as that of the fourth embodiment. This embodiment is directed to still another structure that allows a solution to easily reach even a probe electrode E with a small area as well while reliably covering steps.

As shown in FIG. 15, a planarization layer 242 having a flat upper surface is formed on a structure 240 having a circuit C built in a circuit area CA. A metal film 222 which is patterned to form a probe electrode, and a passivation film 244 which covers an electrode area EA and the circuit area CA are stacked on the planarization layer 242. Namely, the nucleic acid detection device has the planarization layer 242 formed under the metal film 222 and passivation film 244 and having the flat upper surface. The planarization layer 242 includes a metal portion 242a which electrically connects the circuit C to the metal film 222.

The planarization layer 242 reliably covers steps on the surface of the circuit area CA of the structure 240. The metal film 222 is stacked on the flat upper surface of the planarization layer 242. After patterning the metal film 222, the passivation film 244 is stacked on the metal film 222. Thus, the passivation film 244 only need cover the metal film 222 reliably. Accordingly, the passivation film 244 may be a thin film. This allows the solution to reach the probe electrode E easily.

The nucleic acid detection device according to this embodiment is fabricated in the following manner.

A semiconductor substrate including a circuit, or a substrate made of glass, resin, ceramics, metal, or the like and having steps is subjected to a planarization process to decrease the steps. The planarization technique is not particularly specified, and may be etching using a solution or a gas, or mechanical polishing such as the CMP process (Chemical Mechanical Planarization). After the planarization, a metal thin film is formed on the planarization layer by sputtering. Alternatively, the metal thin film may be formed by deposition, printing, or the like. After that, wiring portions, pad portions, and the like are patterned by etching where necessary. After that, a passivation film is formed by coating. Alternatively, the passivation film may be formed by sputtering, deposition, printing, or the like. After that, the passivation film is selectively etched to form openings, so the metal thin film is partly exposed through the openings to serve as electrodes. The technique to form the openings is not particularly specified. The planarization process allows use of a thinner passivation film.

After that, nucleic acid probes are immobilized to form probe electrodes in the same manner as in the first embodiment.

The nucleic acid detection device according to this embodiment is used in the following manner.

A solution containing target nucleic acids is supplied to the flow channel to cause a hybridization reaction. As the passivation film which defines the probe electrodes is thin, the target nucleic acids contained in the solution are supplied to the probe electrodes efficiently. Thus, even a small amount of nucleic acids sufficiently allows a hybridization reaction.

After that, washing, reaction with the nucleic acid recognition bodies, and measurement of an electrochemical reaction are performed in the same manner as in the first embodiment to detect nucleic acids.

### EXAMPLE

An example that uses the nucleic acid detection device according to the third embodiment will be described in detail.

1. Preparation of Nucleic Acid Detection Device

A nucleic acid detection device 1 with a structure that satisfied the requirements described in the third embodiment and a nucleic acid detection device 2 with a structure that did not satisfy them were fabricated. In each of the nucleic acid detection devices 1 and 2, electrodes having different areas are formed two for each area.

Nucleic acid probes having the following sequences were immobilized to the respective electrodes of each area of the nucleic acid detection devices 1 and 2.

Probe A: gtttctgcac ccgga

Probe B: gtttctgcgc ccgga

Flow channels were similarly formed in the nucleic acid detection devices 1 and 2 having the immobilized nucleic acid probes.

2. Respective Types of Nucleic Acid Reactions

A sample nucleic acid having the following sequence including that complementary with the probe A was used.

Sample Nucleic Acid: ggcctccgct ctcgcttcgc ctctttcacc ccgcgcccag ccccgccccg cgccgcgaag aaatgaaact cacagaccct gtgctgaggg cggctccggg cgcagaaacg aaacctagct

A 2×SSC solution containing the sample nucleic acid was supplied to the flow channels of the respective nucleic acid detection devices 1 and 2 to fill the electrodes. The devices 1 and 2 were then set still at 35° C. for 60 min to cause a hybridization reaction of the sample nucleic acid with the nucleic acid probes. Subsequently, a 0.2×SSC solution was supplied to the respective flow channels to fill the electrodes. The devices 1 and 2 were then set still at 35° C. or 60 min to cause a washing reaction. Subsequently, a phosphoric acid solution containing 50 μM of Hoechst 33258 as a nucleic acid detection body was supplied to the respective flow channels to fill the electrodes, to cause the nucleic acid and Hoechst 33258 to interact with each other.

3. Electrochemical Measurement

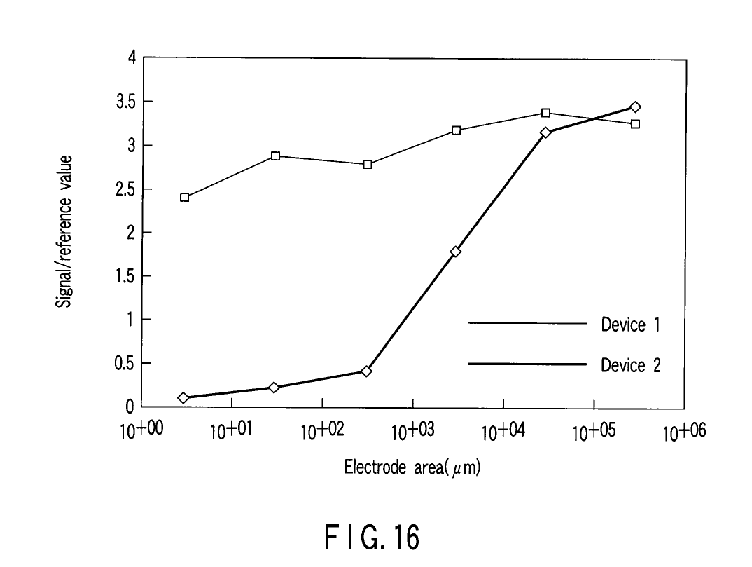

FIG. 16 shows the result of electrochemical measurement. The axis of abscissa represents the electrode area. Values obtained by dividing a current value obtained by an electrode to which the probe A is immobilized, by a current value obtained by an electrode to which the probe B is immobilized are plotted along the axis of ordinate. With the nucleic acid detection device 2, as the area of the nucleic acid detection electrodes decreases, signal increase decreases, so that detection of the nucleic acid is difficult. The nucleic acid detection device 1 allows the nucleic acid to be detected even when the area of the nucleic acid detection electrodes is small.

The embodiments of the present invention are described above with reference to the accompanying drawings. Note that the present invention is not limited to the above embodiments. Various modifications and changes may be made without departing from the spirit and scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A device used to detect a nucleic acid utilizing an electrochemical reaction, comprising:
a flow channel through which a solution containing at least one of a target nucleic acid and a nucleic acid recognition body flows, and which includes a flat bottom surface parallel to a flowing direction of the solution and an inclined surface gradually projecting from the bottom surface in the flowing direction; and
a probe electrode provided on the inclined surface and formed of an exposed portion of a metal film through an opening of a passivation film stacked on the metal film;
an angle θ that the probe electrode forms with respect to the bottom surface, a maximal width R of the opening in the flowing direction, and a thickness d of the passivation film satisfying
tan θ>d/R

2. A device according to claim 1, further comprising a plurality of probe electrodes similar in structure to the probe electrode and including a probe electrode differing in area from the probe electrode.

3. A device used to detect a nucleic acid utilizing an electrochemical reaction, comprising:
a flow channel through which a solution containing at least one of a target nucleic acid and a nucleic acid recognition body flows; and
a probe electrode provided in the flow channel and formed of an exposed portion of a metal film through an opening of a passivation film stacked on the metal film;
the passivation film satisfying
d2>d1
where d1 is a thickness of the passivation film upstream in a flowing direction of the solution, and d2 is a thickness of the passivation film downstream in the flowing direction of the solution.

4. A device according to claim 3, further comprising a plurality of probe electrodes similar in structure to the probe electrode and including a probe electrode differing in area from the probe electrode.

5. A device used to detect a nucleic acid utilizing an electrochemical reaction, comprising:
a flow channel through which a solution containing at least one of a target nucleic acid and a nucleic acid recognition body flows and which includes a flat bottom surface parallel to a flowing direction of the solution and a projection including a projecting surface projecting from the bottom surface; and
a probe electrode provided on the projecting surface and formed of an exposed portion of a metal film through an opening of a passivation film stacked on the metal film;
a distance L from an end of the projection upstream in the flowing direction of the solution to the probe electrode satisfying
L<0.065×Re×D
Re=ρuD/μ
D=4S/Lp
where ρ is a density of the solution, u is a flow velocity of the solution, μ is a viscosity of the solution, S is a sectional area of the flow channel, and Lp is a wall peripheral perimeter of the flow channel.

6. A device according to claim 5, further comprising a plurality of probe electrodes similar in structure to the probe electrode and including a probe electrode differing in area from the probe electrode.

7. A device used to detect a nucleic acid utilizing an electrochemical reaction, comprising:
a flow channel through which a solution containing at least one of a target nucleic acid and a nucleic acid recognition body flows; and
a probe electrode provided in the flow channel and formed of an exposed portion of a metal film through an opening of a passivation film stacked on the metal film;
the passivation film including a first portion which covers an electrode area surrounding the probe electrode, and a second portion which covers an area different from the electrode area, and the first portion being thinner than the second portion.

8. A device according to claim 7, further comprising a plurality of probe electrodes similar in structure to the probe electrode, and wherein the passivation film further include a plurality of first portions similar in structure to the first portion, and the first portions are arranged through the second portion.

9. A device according to claim 7, further comprising a plurality of probe electrodes similar in structure to the probe electrode and including a probe electrode differing in area from the probe electrode.

10. A device used to detect a nucleic acid utilizing an electrochemical reaction, comprising:
a flow channel through which a solution containing at least one of a target nucleic acid and a nucleic acid recognition body flows;
a probe electrode provided in the flow channel and formed of an exposed portion of a metal film through an opening of a passivation film stacked on the metal film; and
a planarization layer provided under the metal film and the passivation film and including a flat upper surface.

11. A device according to claim 10, further comprising a plurality of probe electrodes similar in structure to the probe electrode and including a probe electrode differing in area from the probe electrode.
